(19) United States
(12) Patent Application Publication
(10) Pub. No.: US 2010/0074953 A1
(43) Pub. Date: Mar. 25, 2010

(54) Methods for intiating in situ formation of hydrogels

(76) Inventors: Hassan Chaouk, Smyrna, GA (US); Bruktawit Asfaw, Norcross, GA (US)

Correspondence Address: LAW OFFICE OF COLLEN A. BEARD, LLC, P. O. BOX 1064, DECATUR, GA, 30031-1064, US

(21) Appl. No.: 12/592,305

(22) Filed: Nov. 23, 2009

Related U.S. Application Data
(62) Division of application No. 10/295,817, filed on Nov. 15, 2002, now abandoned.
(60) Provisional application No. 60/332,448, filed on Nov. 16, 2001.

(51) Int. Cl. A61K 9/00; A61K 31/74; A61P 9/00

(52) U.S. Cl. 424/484; 424/78.08

(57) ABSTRACT

Methods for initiating formation of hydrogels in situ from a gellable composition and an initiator where the initiator is provided as a solid article or is contained in a solution infused to the intended site of formation of the hydrogel.

### PRIORITY CLAIM

This application claims priority to U.S. provisional application Ser. No. 60/332,448 filed on Nov. 16, 2001.

### BACKGROUND OF THE INVENTION

The invention relates to methods of initiating in situ formation of hydrogels. The invention also relates to initiator systems for initiation of formation of hydrogels.

In one embodiment, the invention relates to methods and initiator systems for initiation of polymerization of polymerizable macromolecular monomers (termed “macromers” or “prepolymers” herein) in situ to form a hydrogel medical device.

Hydrogels are useful for a number of biomedical applications. In situ forming hydrogels are administered to the body in liquid form, whereupon they transform into the solid hydrogel. In situ forming hydrogels are especially useful for some applications, such as embolotherapy, tissue bulking, and drug delivery. In situ forming hydrogels are of several types. One type of in situ forming hydrogels is made from crosslinking macromers. Such macromers contain crosslinkable groups that can be crosslinked after administration (in situ) to form the hydrogel. See WO 01/68720 to BioCure, Inc. and U.S. Pat. No. 5,410,016 to Hubbell et al.

WO 01/68720 describes a two part macromer system used to form a hydrogel in situ. Each of the two parts includes a redox couple. When the two parts are combined, crosslinking (formation of the hydrogel) begins. It is sometimes preferable to begin this crosslinking at the intended site of application. Accordingly, the two parts are not combined until they are both applied to the intended site. Premature mixing of the two parts can lead to unintended, premature formation of the hydrogel (and clogging of the catheter, for example). A dual lumen catheter can be used to deliver the macromer composition through one lumen and the initiator through the second lumen (or to deliver the two redox components through separate lumens). However, dual lumen catheters face restrictions in terms of size—they cannot be made below a certain diameter and maintain the needed flexibility to access tortuous or otherwise hard to reach sites—such as, particularly, sites in the neurovascular vasculature.

Embolic agents are useful for a variety of bioapplications, such as occluding blood vessels, occluding other body lumens such as fallopian tubes, filling aneurysm sacs, as arterial sealants, and as puncture sealants. Embolization of blood vessels is performed for a number of reasons, e.g. to reduce blood flow to and encourage atrophy of tumors, such as in the liver, to reduce blood flow and induce atrophy of uterine fibroids, for treatment of vascular malformations, such as arteriovenous malformations (AVMs) and arteriovenous fistulas (AVFs), to seal endoleaks into aneurysm sacs, to stop uncontrolled bleeding, or to slow bleeding prior to surgery.

Gynecologic embolotherapy may be conducted for a variety of purposes including the treatment of uterine fibroids, the treatment of postpartum and post caesarean bleeding, the treatment of post surgical vaginal bleeding, the prevention and/or treatment of hemorrhage from ectopic pregnancy, prophylactically prior to myomectomy and in obstetrical patients at high risk for bleeding, such as those patients with placenta previa, placenta accreta, uterine fibroids, and twin fetal death.

Abdominal aortic aneurysms (AAA) and thoracic aortic aneurysms (TAA) are relatively rare but often fatal conditions. Open surgery, primarily using clips or ligation techniques, has been the traditional means of treating AAAs and TAAs. Endovascular techniques, i.e. the placement of a stent graft at the site of the aneurysm, have become more popular. The currently available stent graft products, however, are not well matched to the unpredictable and singular anatomy presented by the aneurysm and its surrounding vasculature. Often, there are leaks into the excluded aneurysm sac, termed endoleaks, due to several reasons, including feeder vessels into the sac, spaces between the stent graft and the vessel wall, or holes in the stent graft wall. Such endoleaks can cause the pressure within the aneurysm sac to increase and cause the aneurysm to further expand and to rupture. Various embolic materials, including the devices and materials discussed above, have been placed in the aneurysm sac to induce thrombosis or otherwise to pack the aneurysm sac to seal the endoleak. Embolic materials are also used to occlude feeder vessels into the sac. WO 00/56380 to Micro Therapeutics, Inc. discloses the use of precipitating polymers and prepolymers such as cyanoacrylate to seal endoleaks.

There are many instances in which an appropriate hydrogel biomaterial is needed for use in repair of tissues and in augmentation of tissues. Applications for an appropriate hydrogel biomaterial include repair of defects and conditions in a tissue caused by disease, injury, or aging, repair of congenital defects and conditions in a tissue, and augmentation of tissues to provide a desirable functional, reconstructive, or cosmetic change. Hydrogel biomaterials are also needed for sealing tissues to prevent post operation leakage, for tissue adherence, and for prevention of tissue adhesion. Hydrogel biomaterials are also needed for cell encapsulation for forming bioreactors, for example, and for cell implantation.

Gastroesophageal reflux is a physical condition in which stomach acids reflux, or flow back from the stomach into the esophagus. Frequent reflux episodes (two or more times per week), may result in a more severe problem known as gastroesophageal reflux disease (GERD). The primary cause of GERD is believed to be the lack of competency of the lower esophageal sphincter. The lower esophageal sphincter, or valve, is comprised of smooth muscle located at the gastroesophageal (GE) junction and functions to allow food and liquid to pass into the stomach but prevent regurgitation of stomach contents. Bulking of the lower esophageal sphincter may be beneficial.

Vesicoureteral reflux is a condition wherein there is an abnormal development of the ureteral bud as it enters the bladder during embryologic development. The shortened course of the ureter through the bladder musculature decreases the ureteral resistance and allows for urine to reflux from the bladder reservoir back up into the ureter and into the kidney. Vesicoureteral reflux can be treated by endoscopic injection of a bulking agent in the submucosal space. Generally, a cystoscope is inserted into the bladder, a needle is inserted through the cystoscope and placed under direct vision underneath the refluxing ureter in the submucosal space, and the bulking agent is injected until the gaping ureteric orifice configuration changes into a half-moon slit.

Urinary incontinence is the inability to retain urine and not void urine involuntarily. As a person ages, his ability to voluntarily control the sphincter muscle is lost in the same way that general muscle tone deteriorates with age. This can also occur when a radical event such as paraplegia “disconnects” the parasympathetic nervous system causing a loss of sphincter control. Some types of incontinence can be treated by injection of a bulking agent into the submucosa of the urethra, in order to “beef up” the area and improve muscle tone.

Hydrogel biomaterials are used in a number of applications in the field of plastic and reconstructive surgery. For example, various compositions have been used for implantation in the lips and to fill in wrinkles. Hydrogel biomaterials have also been used as breast implants, typically encased within a silicone shell.

Hydrogel biomaterials have been used in repair of hard tissue such as cartilage and bone. Musculoskeletal damage can occur due to injury or decay and can be repaired, in some cases, by replacement of the damaged tissue with an appropriate biomaterial.

Accordingly, hydrogel biomaterials are desired for many applications and alternate in situ forming hydrogels would be useful.

### DETAILED DESCRIPTION OF THE INVENTION

The invention relates to methods of initiating formation of hydrogels in situ to form a hydrogel medical device. The invention also relates to initiator systems for initiating formation of hydrogels in situ to form a hydrogel medical device.

Hydrogels

The hydrogel can be any of a number of types that are biocompatible and that form in response to an initiator. The hydrogel is formed from a composition including polymers or macromers that are curable, meaning that they can be cured or otherwise modified, in situ, at the tissue site, in response to an initiator, and undergo a phase or chemical change sufficient to retain a desired position and configuration. Examples include hydrogels formed from macromers, as described in WO 01/68720 to BioCure, Inc. and U.S. Pat. No. 5,410,016 to Hubbell et al. The term “gellable composition” is used herein to refer to the polymeric or macromeric composition that forms the hydrogel in response to initiation.

A gellable composition, formed from the polymers or macromers, and optionally other components, is deliverable to the intended site of application. The properties, i.e. viscosity, of this composition will vary depending upon the intended final use of the composition. For example, a composition intended for use as an embolic device will have certain desired characteristics. The composition is delivered to the intended site through an appropriate delivery device, such as a catheter or syringe. Before, during, or after delivery, the composition is exposed to the initiator system, causing gellation of the polymers or macromers and formation of the hydrogel device.

Gellation Mechanisms

Gellation of the polymers or macromers can be via a number of mechanisms, such as physical crosslinking or chemical crosslinking. Physical crosslinking includes, but is not limited to, complexation, hydrogen bonding, desolvation, Van der wals interactions, and ionic bonding. Chemical crosslinking can be accomplished by a number of means including, but not limited to, chain reaction (addition) polymerization, step reaction (condensation) polymerization and other methods of increasing the molecular weight of polymers/oligomers to very high molecular weights. Chain reaction polymerization includes, but is not limited to, free radical polymerization (thermal, photo, redox, atom transfer polymerization, etc.), cationic polymerization (including onium), anionic polymerization (including group transfer polymerization), certain types of coordination polymerization, certain types of ring opening and metathesis polymerizations, etc. Step reaction polymerizations include all polymerizations which follow step growth kinetics including but not limited to reactions of nucleophiles with electrophiles, certain types of coordination polymerization, certain types of ring opening and metathesis polymerizations, etc. Other methods of increasing molecular weight of polymers/oligomers include but are not limited to polyelectrolyte formation, grafting, ionic crosslinking, etc.

Various crosslinkable groups are known to those skilled in the art and can be used, according to what type of crosslinking is desired. For example, hydrogels can be formed by the ionic interaction of divalent cationic metal ions (such as Ca⁺² and Mg⁺²) with ionic polysaccharides such as alginates, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, and amylopectin. Multifunctional cationic polymers, such as poly(l-lysine), poly(allylamine), poly(ethyleneimine), poly(guanidine), poly(vinyl amine), which contain a plurality of amine functionalities along the backbone, may be used to further induce ionic crosslinks.

Hydrophobic interactions are often able to induce physical entanglement, especially in polymers, that induces increases in viscosity, precipitation, or gelation of polymeric solutions. Block and graft copolymers of water soluble and insoluble polymers exhibit such effects, for example, poly(oxyethylene)-poly(oxypropylene) block copolymers, copolymers of poly(oxyethylene) with poly(styrene), poly(caprolactone), poly(butadiene), etc.

Other means for gellation also may be advantageously used with macromers that contain groups that demonstrate activity towards functional groups such as amines, imines, thiols, carboxyls, isocyanates, urethanes, amides, thiocyanates, hydroxyls, etc.

Desirable crosslinkable groups include (meth)acrylamide, (meth)acrylate, styryl, vinyl ester, vinyl ketone, vinyl ethers, etc. Particularly desirable are ethylenically unsaturated functional groups.

Macromer Systems

The hydrogel can be formed from one or more macromers that include a hydrophilic or water soluble region and one or more crosslinkable regions. The macromers may also include other elements such as one or more degradable or biodegradable regions. A variety of factors—primarily the desired characteristics of the formed hydrogel—determines the most appropriate macromers to use. Many macromer systems that form biocompatible hydrogels can be used.

Macromers suitable for use in the compositions described herein are disclosed in WO 01/68720 to BioCure, Inc. Other suitable macromers include those disclosed in U.S. Pat. No. 5,410,016 to Hubbell et al., U.S. Pat. No. 4,938,763 to Dunn et al., U.S. Pat. Nos. 5,100,992 and 4,826,945 to Cohn et al., U.S. Pat. Nos. 4,741,872 and 5,160,745 to De Luca et al, and U.S. Pat. No. 4,511,478 to Nowinski et al.

The Macromer Backbone

Macromers can be constructed from a number of hydrophilic polymers, such as, but not limited to, polyvinyl alcohols (PVA), polyethylene glycols (PEG), polyvinyl pyrrolidone (PVP), polyalkyl hydroxy acrylates and methacrylates (e.g. hydroxyethyl methacrylate (HEMA), hydroxybutyl methacrylate (HBMA), and dimethylaminoethyl methacrylate (DMEMA)), polysaccharides (e.g. cellulose, dextran), polyacrylic acid, polyamino acids (e.g. polylysine, polyethylmine, PAMAM dendrimers), polyacrylamides (e.g. polydimethylacrylamid-co-HEMA, polydimethylacrylamid-co-HBMA, polydimethylacrylamid-co-DMEMA). The macromers can be linear or can have a branched, hyperbranched, or dendritic structure.

In one preferred embodiment, the macromers have a backbone of a polymer comprising units having a 1,2-diol or 1,3-diol structure, such as polyhydroxy polymers. For example, polyvinyl alcohol (PVA) or copolymers of vinyl alcohol contain a 1,3-diol skeleton. The backbone can also contain hydroxyl groups in the form of 1,2-glycols, such as copolymer units of 1,2-dihydroxyethylene. These can be obtained, for example, by alkaline hydrolysis of vinyl acetate-vinylene carbonate copolymers. Other polymeric diols can be used, such as saccharides.

In addition, the macromers can also contain small proportions, for example, up to 20%, preferably up to 5%, of comonomer units of ethylene, propylene, acrylamide, methacrylamide, dimethacrylamide, hydroxyethyl methacrylate, alkyl methacrylates, alkyl methacrylates which are substituted by hydrophilic groups, such as hydroxyl, carboxyl or amino groups, methyl acrylate, ethyl acrylate, vinylpyrrolidone, hydroxyethyl acrylate, allyl alcohol, styrene, polyalkylene glycols, or similar comonomers usually used.

Polyvinyl alcohols that can be used as macromer backbones include commercially available PVAs, for example Vinol® 107 from Air Products (MW 22,000 to 31,000, 98 to 98.8% hydrolyzed), Polysciences 4397 (MW 25,000, 98.5% hydrolyzed), BF 14 from Chan Chun, Elvanol® 90-50 from DuPont and UF-120 from Unitika. Other producers are, for example, Nippon Gohsei (Gohsenol®), Monsanto (Gelvatol®), Wacker (Polyviol®), Kuraray, Deriki, and Shin-Etsu. In some cases it is advantageous to use Mowiol® products from Hoechst, in particular those of the 3-83, 4-88, 4-98, 6-88, 6-98, 8-88, 8-98, 10-98, 20-98, 26-88, and 40-88 types.

It is also possible to use copolymers of hydrolyzed or partially hydrolyzed vinyl acetate, which are obtainable, for example, as hydrolyzed ethylene-vinyl acetate (EVA), or vinyl chloride-vinyl acetate, N-vinylpyrrolidone-vinyl acetate, and maleic anhydride-vinyl acetate. If the macromer backbones are, for example, copolymers of vinyl acetate and vinylpyrrolidone, it is again possible to use commercially available copolymers, for example the commercial products available under the name Luviskol® from BASF. Particular examples are Luviskol VA 37 HM, Luviskol VA 37 E and Luviskol VA 28. If the macromer backbones are polyvinyl acetates, Mowilith 30 from Hoechst is particularly suitable.

Polyvinyl alcohols that can be derivatized as described herein preferably have a molecular weight of at least about 2,000. As an upper limit, the PVA may have a molecular weight of up to 1,000,000. Preferably, the PVA has a molecular weight of up to 300,000, especially up to approximately 130,000, and especially preferably up to approximately 60,000.

The PVA usually has a poly(2-hydroxy)ethylene structure. The PVA derivatized in accordance with the disclosure may, however, also comprise hydroxy groups in the form of 1,2-glycols.

The PVA system can be a fully hydrolyzed PVA, with all repeating groups being —CH₂—CH(OH), or a partially hydrolyzed PVA with varying proportions (1% to 25%) of pendant ester groups. PVA with pendant ester groups have repeating groups of the structure CH₂—CH(OR) where R is COCH₃ group or longer alkyls, as long as the water solubility of the PVA is preserved. The ester groups can also be substituted by acetaldehyde or butyraldehyde acetals that impart a certain degree of hydrophobicity and strength to the PVA. For an application that requires an oxidatively stable PVA, the commercially available PVA can be broken down by NaIO₄—KMnO₄ oxidation to yield a small molecular weight (2000 to 4000) PVA.

The PVA is prepared by basic or acidic, partial or virtually complete hydrolysis of polyvinyl acetate. In a preferred embodiment, the PVA comprises less than 50% of vinyl acetate units, especially less than about 25% of vinyl acetate units. Preferred amounts of residual acetate units in the PVA, based on the sum of vinyl alcohol units and acetate units, are approximately from 3 to 25%.

Crosslinkable Groups

The macromers have at least two pendant chains containing groups that can be crosslinked. The term group includes single polymerizable moieties, such as an acrylate, as well as larger crosslinkable regions, such as oligomeric or polymeric regions. The crosslinkers are desirably present in an amount of from approximately 0.01 to 10 milliequivalents of crosslinker per gram of backbone (meq/g), more desirably about 0.05 to 1.5 meq/g. The macromers can contain more than one type of crosslinkable group. The crosslinkable groups require an initiator to crosslink, that is, they do not spontaneously crosslink under the conditions employed.

In the embodiment where the macromer backbone comprises a polyhydroxy compound, the pendant chains can be attached via the hydroxyl groups of the polymer backbone. Desirably, the pendant chains having crosslinkable groups are attached via cyclic acetal linkages to 1,2-diol or 1,3-diol hydroxyl groups.

Ethylenically unsaturated groups can be crosslinked via free radical initiated polymerization, including via photoinitiation, redox initiation, and thermal initiation. Systems employing these means of initiation are well known to those skilled in the art. In one embodiment, a two part redox system is employed. One part of the system contains a reducing agent such as a ferrous salt. Various ferrous salts can be used, such as, for example, ferrous gluconate dihydrate, ferrous lactate dihydrate, or ferrous acetate. The other half of the system contains an oxidizing agent such as hydrogen peroxide.

Other reducing agents can be used, such as, but not limited to, cuprous salts, cerous salts, cobaltous salts, permanganate, and manganous salts. Ascorbate, for example, can be used as a coreductant to recycle the reductant and reduce the amount needed. This can reduce the toxicity of a ferrous based system. Other oxidizing agents that can be used include, but are not limited to, t-butyl hydroperoxide, t-butyl peroxide, benzoyl peroxide, cumyl peroxide, etc.

Initiation Systems

The term “initiator” is used herein to refer to an element which begins the process of gelation of a gellable composition. In some cases, the term “initiator” as used herein refers to one part of an initiator system. For example, a redox couple may be used as the initiator system, wherein one part of the couple is included in the gellable composition and the other part of the couple is separately provided. The part of the couple separately provided is referred to as the “initiator” herein.

In one embodiment of the invention, the initiator is provided at the site in the form of a solid article. Examples of solid articles that can be or provide the initiator are microspheres, disks, coils, and other shaped articles. The solid article can be made of metal, such as a metallic coil, or a polymer, such as polymeric microspheres.

There are many ways in which the solid article can embody the initiator. For example, the article can be made entirely or partially of the initiator, the initiator can be coated on the surface of the article, or the initiator can be embedded or impregnated into the article. For example, the solid article could be microspheres or a solid disk made from an initiator. The initiator can be released from the solid article, or simply contact with the solid article can provide initiation.

The solid article initiator is delivered to the site where the hydrogel article is to be formed. It can be delivered before, during, or after the gellable composition is delivered. As one example, the solid initiator could be an embolic coil coated with initiator that is placed in an aneurysm prior to delivery of gellable prepolymer to the aneurysm. The initiator could be microspheres impregnated with an initiator compound that are injected to a site to be bulked after the gellable composition has been delivered to the site. As another example, the initiator could be a polymer sheet coated with initiator compound that is applied to an area to be sealed, prior to application of the gellable composition to the area.

In a case where crosslinkable groups are initiated by free radical polymerization, one part of a redox couple can be delivered along with the macromer solution through a single lumen catheter and the other part of the redox couple can be delivered through the solid article(s). Other types of initiators can also be supplied via a solid article, such as divalent cationic ions for ionic crosslinking of polysaccharides.

In another embodiment of the invention, the initiator is provided as an infusion of a solution containing the initiator. The infusion solution can be provided via a separate access point, or can be provided via the same access point, but downstream of the gellable composition. For example, in the case of embolic agent delivery to a neurovascular aneurysm, the gellable composition can be delivered via a catheter introduced via the femoral artery, as is standard in practice, while the initiator infusion solution can be delivered via a catheter introduced via the carotid artery. In another embodiment, a dual lumen catheter can be employed wherein one lumen extends further than the other so that the catheter diameter is narrower at its distal end (and can access smaller vasculature). The lumens can be arranged coaxially or side by side. The gellable composition is delivered via the shorter lumen, while the initiator infusion solution is delivered via the longer lumen. If the lumens are arranged coaxially, the longer lumen is the internal lumen.

In the example of macromers crosslinked via free radical chemistry using a redox initiator, the macromer solution containing one part of the redox couple can be delivered through a catheter introduced via the femoral artery and the other part of the redox couple can be delivered through a catheter introduced via the carotid artery.

In another embodiment, the initiator system is provided at the delivery tip of the catheter. For example, the catheter tip could provide (deliver) one part of a redox couple while the other part of the couple is provided in a solution of the gellable composition.

Specific Macromers

Specific macromers that are suitable for use in the embolic compositions are disclosed in U.S. Pat. Nos. 5,508,317, 5,665,840, 5,807,927, 5,849,841, 5,932,674, 5,939,489, and 6,011,077.

In one embodiment, units containing a crosslinkable group conform, in particular, to the formula I

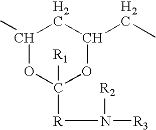

in which R is a linear or branched C₁-C₈ alkylene or a linear or branched C₁-C₁₂ alkane. Suitable alkylene examples include octylene, hexylene, pentylene, butylene, propylene, ethylene, methylene, 2-propylene, 2-butylene and 3-pentylene. Preferably lower alkylene R has up to 6 and especially preferably up to 4 carbon atoms. The groups ethylene and butylene are especially preferred. Alkanes include, in particular, methane, ethane, n- or isopropane, n-, sec- or tert-butane, n- or isopentane, hexane, heptane, or octane. Preferred groups contain one to four carbon atoms, in particular one carbon atom.

R₁ is hydrogen, a C₁-C₆ alkyl, or a cycloalkyl, for example, methyl, ethyl, propyl or butyl and R₂ is hydrogen or a C₁-C₆ alkyl, for example, methyl, ethyl, propyl or butyl. R₁ and R₂ are preferably each hydrogen.

R₃ is an olefinically unsaturated electron attracting copolymerizable radical having up to 25 carbon atoms. In one embodiment, R₃ has the structure

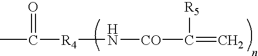

where R₄ is the

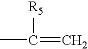

group if n=zero, or the

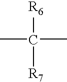

bridge if n=1;
R₅ is hydrogen or C₁-C₄ alkyl, for example, n-butyl, n- or isopropyl, ethyl, or methyl;
n is zero or 1, preferably zero; and
R₆ and R₇, independently of one another, are hydrogen, a linear or branched C₁-C₈ alkyl, aryl or cyclohexyl, for example one of the following: octyl, hexyl, pentyl, butyl, propyl, ethyl, methyl, 2-propyl, 2-butyl or 3-pentyl. R₆ is preferably hydrogen or the CH₃ group, and R₇ is preferably a C₁-C₄ alkyl group. R₆ and R₇ as aryl are preferably phenyl.

In another embodiment, R₃ is an olefinically unsaturated acyl group of formula R₈—CO—, in which R₈ is an olefinically unsaturated copolymerizable group having from 2 to 24 carbon atoms, preferably from 2 to 8 carbon atoms, especially preferably from 2 to 4 carbon atoms. The olefinically unsaturated copolymerizable radical R₈ having from 2 to 24 carbon atoms is preferably alkenyl having from 2 to 24 carbon atoms, especially alkenyl having from 2 to 8 carbon atoms and especially preferably alkenyl having from 2 to 4 carbon atoms, for example ethenyl, 2-propenyl, 3-propenyl, 2-butenyl, hexenyl, octenyl or dodecenyl. The groups ethenyl and 2-propenyl are preferred, so that the group —CO—R₈ is the acyl radical of acrylic or methacrylic acid.

In another embodiment, the group R₃ is a radical of formula

—[CO—NH—(R₉—NH—CO—O)_{q}—R₁₀—O]ₚ—CO—R₈

wherein p and q are zero or one and
R₉ and R₁₀ are each independently lower alkylene having from 2 to 8 carbon atoms, arylene having from 6 to 12 carbon atoms, a saturated divalent cycloaliphatic group having from 6 to 10 carbon atoms, arylenealkylene or alkylenearylene having from 7 to 14 carbon atoms or arylenealkylenearylene having from 13 to 16 carbon atoms, and
R₈ is as defined above.

Lower alkylene R₉ or R₁₀ preferably has from 2 to 6 carbon atoms and is especially straight-chained. Suitable examples include propylene, butylene, hexylene, dimethylethylene and, especially preferably, ethylene.

Arylene R₉ or R₁₀ is preferably phenylene that is unsubstituted or is substituted by lower alkyl or lower alkoxy, especially 1,3-phenylene or 1,4-phenylene or methyl-1,4-phenylene.

A saturated divalent cycloaliphatic group R₉ or R₁₀ is preferably cyclohexylene or cyclohexylene-lower alkylene, for example cyclohexylenemethylene, that is unsubstituted or is substituted by one or more methyl groups, such as, for example, trimethylcyclohexylenemethylene, for example the divalent isophorone radical.

The arylene unit of alkylenearylene or arylenealkylene R₉ or R₁₀ is preferably phenylene, unsubstituted or substituted by lower alkyl or lower alkoxy, and the alkylene unit thereof is preferably lower alkylene, such as methylene or ethylene, especially methylene. Such radicals R₉ or R₁₀ are therefore preferably phenylenemethylene or methylenephenylene.

Arylenealkylenearylene R₉ or R₁₀ is preferably phenylene-lower alkylene-phenylene having up to 4 carbon atoms in the alkylene unit, for example phenyleneethylenephenylene.

The groups R₉ and R₁₀ are each independently preferably lower alkylene having from 2 to 6 carbon atoms, phenylene, unsubstituted or substituted by lower alkyl, cyclohexylene or cyclohexylene-lower alkylene, unsubstituted or substituted by lower alkyl, phenylene-lower alkylene, lower alkylene-phenylene or phenylene-lower alkylene-phenylene.

The group —R₉—NH—CO—O— is present when q is one and absent when q is zero. Macromers in which q is zero are preferred.

The group —CO—NH—(R₉—NH—CO—O)_{q}—R₁₀—O— is present when p is one and absent when p is zero. Macromers in which p is zero are preferred.

In macromers in which p is one, q is preferably zero. Macromers in which p is one, q is zero, and R₁₀ is lower alkylene are especially preferred.

All of the above groups can be monosubstituted or polysubstituted, examples of suitable substituents being the following: C₁-C₄ alkyl, such as methyl, ethyl or propyl, —COOH, —OH, —SH, C₁-C₄ alkoxy (such as methoxy, ethoxy, propoxy, butoxy, or isobutoxy), —NO₂, —NH₂, —NH(C₁-C₄), —NH—CO—NH₂, —N(C₁-C₄ alkyl)₂, phenyl (unsubstituted or substituted by, for example, —OH or halogen, such as Cl, Br or especially I), —S(C₁-C₄ alkyl), a 5- or 6-membered heterocyclic ring, such as, in particular, indole or imidazole, —NH—C(NH)—NH₂, phenoxyphenyl (unsubstituted or substituted by, for example, —OH or halogen, such as Cl, Br or especially I), an olefinic group, such as ethylene or vinyl, and CO—NH—C(NH)—NH₂.

Preferred substituents are lower alkyl, which here, as elsewhere in this description, is preferably C₁-C₄ alkyl, C₁-C₄ alkoxy, COOH, SH, —NH₂, —NH(C₁-C₄ alkyl), —N(C₁-C₄ alkyl)₂ or halogen. Particular preference is given to C₁-C₄ alkyl, C₁-C₄ alkoxy, COOH and SH.

For the purposes of this invention, cycloalkyl is, in particular, cycloalkyl, and aryl is, in particular, phenyl, unsubstituted or substituted as described above.

Modifiers

The macromers can include further modifier groups and crosslinkable groups. Some such groups are described in U.S. Pat. Nos. 5,508,317, 5,665,840, 5,807,927, 5,849,841, 5,932,674, 5,939,489, and 6,011,077. Crosslinkable groups and the optional further modifier groups can be bonded to the macromer backbone in various ways, for example through a certain percentage of the 1,3-diol units being modified to give a 1,3-dioxane, which contains a crosslinkable group, or a further modifier, in the 2-position. Modifiers that might be attached to the backbone include those to modify the hydrophobicity, active agents or groups to allow attachment of active agents, photoinitiators, modifiers to enhance or reduce adhesiveness, modifiers to impart thermoresponsiveness, modifiers to impart other types of responsiveness, and additional crosslinking groups. These modifiers may be attached to the hydroxyl groups in the backbone, or to other monomeric units included in the backbone.

Attaching a cellular adhesion promoter to the macromers can enhance cellular attachment or adhesiveness of the embolic agents formed by the embolic compositions. These agents are well known to those skilled in the art and include carboxymethyl dextran, proteoglycans, collagen, gelatin, glucosaminoglycans, fibronectin, lectins, polycations, and natural or synthetic biological cell adhesion agents such as RGD peptides.

Having pendant ester groups that are substituted by acetaldehyde or butyraldehyde acetals, for example, can increase the hydrophobicity of the macromers and the formed hydrogel. Hydrophobic groups can desirably be present in an amount from about 0 to 25%.

It may also be desirable to include on the macromer a molecule that allows visualization of the formed hydrogel. Examples include dyes and molecules visualizable by magnetic resonance imaging.

Degradable Regions

The macromers can form a hydrogel that is degradable. Suitable degradable systems are described in WO 01/44307. In the degradable systems described in that application, the macromers include a degradable region in the backbone or on a pendant chain. The degradable region is preferably degradable under in vivo conditions by hydrolysis. The degradable region can be enzymatically degradable. For example, the degradable region may be polymers and oligomers of glycolide, lactide, ε-caprolactone, other hydroxy acids, and other biologically degradable polymers that yield materials that are non-toxic or present as normal metabolites in the body. Preferred poly(α-hydroxy acids) are poly(glycolic acid), poly(DL-lactic acid) and poly(L-lactic acid). Other useful materials include poly(amino acids), poly(anhydrides), poly(orthoesters), poly(phosphazines), and poly(phosphoesters). Polylactones such as poly(ε-caprolactone), poly(ε-caprolactone), poly(δ-valerolactone) and poly(γ-butyrolactone), for example, are also useful. Enzymatically degradable linkages include poly(amino acids), gelatin, chitosan, and carbohydrates. The biodegradable regions may have a degree of polymerization ranging from one up to values that would yield a product that was not substantially water soluble. Thus, monomeric, dimeric, trimeric, oligomeric, and polymeric regions may be used. The biodegradable region could, for example, be a single methacrylate group.

Biodegradable regions can be constructed from polymers or monomers using linkages susceptible to biodegradation, such as ester, acetal, carbonate, peptide, anhydride, orthoester, phosphazine, and phosphoester bonds. The biodegradable regions may be arranged within the macromers such that the formed hydrogel has a range of degradability, both in terms of extent of degradation, whether complete or partial, and in terms of time to complete or partial degradation.

Vinylic Comonomers

The process for polymerization of the macromers may comprise, for example, crosslinking a macromer comprising units of formula I, especially in substantially pure form, that is to say, for example, after single or repeated ultrafiltration, preferably in solution, especially in aqueous solution, in the absence or presence of an additional vinylic comonomer.

The vinylic comonomer may be hydrophilic or hydrophobic, or a mixture of a hydrophobic and a hydrophilic vinylic monomer. Generally, approximately from 0.01 to 80 units of a typical vinylic comonomer react per unit of formula I, especially from 1 to 30 units per unit of formula I, and especially preferably from to 20 units per unit of formula I.

It is also preferable to use a hydrophobic vinylic comonomer or a mixture of a hydrophobic vinylic comonomer with a hydrophilic vinylic comonomer, the mixture comprising at least 50 percent by weight of a hydrophobic vinylic comonomer. In that manner the mechanical properties of the polymer can be improved without the water content falling substantially. In principle, however, both conventional hydrophobic vinylic comonomers and conventional hydrophilic vinylic comonomers are suitable for copolymerization with the macromer.

Suitable hydrophobic vinylic comonomers include, without the list being exhaustive, C₁-C₁₈ alkyl acrylates and methacrylates, C₃-C₁₈ alkyl acrylamides and methacrylamides, acrylonitrile, methacrylonitrile, vinyl-C₁-C₁₈ alkanoates, C₂-C₁₈ alkenes, C₂-C₁₈ haloalkenes, styrene, C₁-C₆ alkylstyrene, vinyl alkyl ethers, in which the alkyl moiety contains from 1 to 6 carbon atoms, C₂-C₁₀ perfluoroalkyl acrylates and methacrylates or correspondingly partially fluorinated acrylates and methacrylates, C₃-C₁₂ perfluoroalkyl-ethylthiocarbonylaminoethyl acrylates and methacrylates, acryloxy- and methacryloxy-alkylsiloxanes, N-vinylcarbazole, C₃-C₁₂ alkyl esters of maleic acid, fumaric acid, itaconic acid, mesaconic acid and the like. C₁-C₄ alkyl esters of vinylically unsaturated carboxylic acids having from 3 to 5 carbon atoms or vinyl esters of carboxylic acids having up to 5 carbon atoms, for example, are preferred.

Examples of suitable hydrophobic vinylic comonomers include methyl acrylate, ethyl acrylate, propyl acrylate, isopropyl acrylate, cyclohexyl acrylate, 2-ethylhexyl acrylate, methyl methacrylate, ethyl methacrylate, propyl methacrylate, vinyl acetate, vinyl propionate, vinyl butyrate, vinyl valerate, styrene, chloroprene, vinyl chloride, vinylidene chloride, acrylonitrile, 1-butene, butadiene, methacrylonitrile, vinyltoluene, vinyl ethyl ether, perfluorohexylethylthiocarbonylaminoethyl methacrylate, isobornyl methacrylate, trifluoroethyl methacrylate, hexafluoroisopropyl methacrylate, hexafluorobutyl methacrylate, tris-trimethylsilyloxy-silyl-propyl methacrylate, 3-methacryloxypropylpentamethyldisiloxane and bis(methacryloxypropyl)tetramethyldisiloxane.

Suitable hydrophilic vinylic comonomers include, without the list being exhaustive, hydroxy-substituted lower alkyl acrylates and methacrylates, acrylamide, methacrylamide, lower alkyl acrylamides and methacrylamides, ethoxylated acrylates and methacrylates, hydroxy-substituted lower alkyl acrylamides and methacrylamides, hydroxy-substituted lower alkyl vinyl ethers, sodium ethylenesulfonate, sodium styrenesulfonate, 2-acrylamido-2-methylpropanesulfonic acid (AMPS® monomer from Lubrizol Corporation), N-vinylpyrrole, N-vinylsuccinimide, N-vinylpyrrolidone, 2- or 4-vinylpyridine, acrylic acid, methacrylic acid, amino- (the term “amino” also including quaternary ammonium), mono-lower alkylamino- or di-lower alkylamino-lower alkyl acrylates and methacrylates, allyl alcohol and the like. Hydroxy-substituted C₂-C₄ alkyl(meth)acrylates, five- to seven-membered N-vinyl lactams, N,N-di-C₁-C₄ alkyl(meth)acrylamides and vinylically unsaturated carboxylic acids having a total of from 3 to 5 carbon atoms, for example, are preferred.

Contrast Agents

It may be desirable to include a contrast agent in the compositions. A contrast agent is a biocompatible (non-toxic) material capable of being monitored by, for example, radiography. The contrast agent can be water soluble or water insoluble. Examples of water soluble contrast agents include metrizamide, iopamidol, iothalamate sodium, iodomide sodium, and meglumine. Iodinated liquid contrast agents include Omnipaque®, Visipaque®, and Hypaque-76®. Examples of water insoluble contrast agents are tantalum, tantalum oxide, barium sulfate, gold, tungsten, and platinum. These are commonly available as particles preferably having a size of about 10 μm or less.

The contrast agent can be added to the compositions prior to administration. Both solid and liquid contrast agents can be simply mixed with a solution of the compositions. Liquid contrast agent can be mixed at a concentration of about 10 to 80 volume percent, more desirably about 20 to 50 volume percent. Solid contrast agents are desirably added in an amount of about 10 to 40 weight percent, more preferably about 20 to 40 weight percent.

Occlusive Devices

It may be desirable to use the compositions in combination with one or more occlusive devices. Such devices include balloons, microcoils, and other devices known to those skilled in the art. The device can be placed at the site to be occluded or filled before, during, or after the composition is administered. For example, an occlusive coil can be placed in an aneurysm sac to be filled and the liquid composition can be injected into the sac to fill the space around the coil. An advantage of using an occlusive device along with the composition is that it may provide greater rigidity to the filling.

Active Agents

An effective amount of one or more biologically active agents can be included in the compositions. It may be desirable to deliver the active agent from the formed hydrogel. Biologically active agents that it may be desirable to deliver include prophylactic, therapeutic, and diagnostic agents including organic and inorganic molecules and cells (collectively referred to herein as an “active agent” or “drug”). A wide variety of active agents can be incorporated into the hydrogel. Release of the incorporated additive from the hydrogel is achieved by diffusion of the agent from the hydrogel, degradation of the hydrogel, and/or degradation of a chemical link coupling the agent to the polymer. In this context, an “effective amount” refers to the amount of active agent required to obtain the desired effect.

Examples of active agents that can be incorporated include, but are not limited to, anti-angiogenic agents, chemotherapeutic agents, radiation delivery devices, such as radioactive seeds for brachytherapy, and gene therapy compositions.

Chemotherapeutic agents that can be incorporated include water soluble chemotherapeutic agents, such as cisplatin (platinol), doxorubicin (adriamycin, rubex), or mitomycin C (mutamycin). Other chemotherapeutic agents include iodinated fatty acid ethyl esters of poppy seed oil, such as lipiodol.

Cells can be incorporated into the compositions, including cells to encourage tissue growth or cells to secrete a desired active agent. For example, cells that can be incorporated include fibroblasts, endothelial cells, muscle cells, stem cells, etc. Cells can be modified to secrete active agents such as growth factors.

Active agents can be incorporated into the compositions simply by mixing the agent with the composition prior to administration. The active agent will then be entrapped in the hydrogel that is formed upon administration of the composition. The active agent can be in compound form or can be in the form of degradable or nondegradable nano or microspheres. It some cases, it may be possible and desirable to attach the active agent to the macromer. The active agent may be released from the macromer or hydrogel over time or in response to an environmental condition.

Other Additives

It may be desirable to include a peroxide stabilizer in redox initiated systems. Examples of peroxide stabilizers are Dequest® products from Solutia Inc., such as for example Dequest® 2010 and Dequest® 2060S. These are phosphonates and chelants that offer stabilization of peroxide systems. Dequest® 2060S is diethylenetriamine penta(methylene phosphonic acid). These can be added in amounts as recommended by the manufacturer.

It may be desirable to include fillers in the compositions, such as fillers that leach out of the formed hydrogel over a period of time and cause the hydrogel to become porous. Such may be desirable, for example, where the composition is used for chemoembolization and it may be desirable to administer a follow up dose of chemoactive agent. Appropriate fillers include calcium salts, for example.

Characteristics that can be Modified

The compositions are highly versatile. A number of characteristics can be easily modified, making the compositions suitable for a number of applications. For example, as discussed above, the polymer backbones can include comonomers to add desired properties, such as, for example, thermoresponsiveness, degradability, gelation speed, and hydrophobicity. Modifiers can be attached to the polymer backbone (or to pendant groups) to add desired properties, such as, for example, thermoresponsiveness, degradability, hydrophobicity, and adhesiveness. Active agents can also be attached to the polymer backbone using the free hydroxyl groups, or can be attached to pendant groups.

The gelation time of the compositions can be varied from about 0.5 seconds to as long as 10 minutes, and longer if desired. The gelation time will generally be affected by, and can be modified by changing at least the following variables: the initiator system, crosslinker density, macromer molecular weight, macromer concentration (solids content), and type of crosslinker. A higher crosslinker density will provide faster gelation time; a lower molecular weight will provide a slower gelation time. A higher solids content will provide faster gelation time. For redox systems the gelation time can be designed by varying the concentrations of the redox components. Higher reductant and higher oxidant will provide faster gelation, higher buffer concentration and lower pH will provide faster gelation.

The firmness of the formed hydrogel will be determined in part by the hydrophilic/hydrophobic balance, where a higher hydrophobic percent provides a firmer hydrogel. The firmness will also be determined by the crosslinker density (higher density provides a firmer hydrogel), the macromer molecular weight (lower MW provides a firmer hydrogel), and the length of the crosslinker (a shorter crosslinker provides a firmer hydrogel).

The swelling of the hydrogel is inversely proportional to the crosslinker density. Generally, no or minimal swelling is desired, desirably less than about 10 percent.

Elasticity of the formed hydrogel can be increased by increasing the size of the backbone between crosslinks and decreasing the crosslinker density. Incomplete crosslinking will also provide a more elastic hydrogel. Preferably the elasticity of the hydrogel substantially matches the elasticity of the tissue to which the composition is to be administered.

Methods of Using the Compositions

The compositions can be used for a number of applications, including embolotherapy, tissue bulking, tissue sealing, drug delivery, etc. In general, the compositions are used by administering the initiator to the intended site and administering the gellable composition to the intended site of administration via any appropriate means, i.e. catheter or syringe. The initiator can be delivered before, during, or after delivery of the gellable composition.

Embolic Compositions

The compositions can be used for a variety of embolotherapy applications such as, but not limited to, vascular occlusion for treatment of tumors or fibroids, occlusion of vascular malformations, such as arteriovenous malformations (AVM), occlusion of the left atrial appendage, fillers for aneurysm sacs, endoleak sealants, arterial sealants, puncture sealants, and occlusion of other lumens such as fallopian tubes.

According to the general method, an effective amount of the gellable composition in an aqueous solvent and the initiator system is administered to the desired site, such as a lumen or an area to be bulked, for example. The term “effective amount”, as used herein, means the quantity of gellable composition needed to fill or block the biological structure of interest. The effective amount of composition administered to a particular patient will vary depending upon a number of factors, including the sex, weight, age, and general health of the patient, the particular site and condition being treated, and the characteristics of the composition and the resulting hydrogel. The composition may be administered over a number of treatment sessions.

In one embodiment, the initiator system in the form of a solid article is delivered to the site prior to the gellable composition. In another embodiment, the initiator system in the form of a solid article can be delivered simultaneously. In another embodiment, a solution containing the initiator is infused through the site while a solution containing the gellable composition is applied to the site.

### EXAMPLES

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

### Example 1

### Solid Article Initiation System

Polyvinyl alcohol based hydrogel microspheres (5 g, 300-500 microns in size) were equilibrated overnight in 20 ml of a 3% hydrogen peroxide solution at room temperature. The microspheres were collected on a 53μ mesh stainless steal sieve and thoroughly washed with saline. The beads were collected and stored in 20 ml of fresh saline.

The gellable composition included a macromer having a PVA backbone (14 kDa, 12% acetate incorporation) modified with 0.45 meq/g N-acrylamidoacetaldehyde dimethyl acetal pendant polymerizable groups (about 6.3 crosslinks per chain). A solution was made of macromer (7% w/w), AMPS (comonomer) (3% w/w), Omnipaque 350 (50% w/w), iron (II) lactate (2000 ppm), ascorbic acid (8.3 mM), and D.I. water.

Two grams of the macromer solution was placed in a 20 ml glass vial. Using a Pasteur pipette, approximately 0.5 ml of microspheres were collected and dispensed into the vial. Upon mixing, an insoluble hydrogel mass was immediately generated.

### Example 2

### Infusion of Initiator

Gelation experiments were performed under flow conditions using a flow model. A sheet of Perspex (7.5×4.5×0.5 inches) contained grooved channels ranging in sizes from 0.192 to 0.064 inches. The sheet was fitted with two inlet ports to allow the insertion of a catheter (1.9 to 5.0 Fr) and the addition of a mobile phase. One outlet port was present. To prevent leakages, the cell was fitted with a cork gasket and screwed to a Perspex sheet of equal dimension. A peristaltic pump was used to deliver the mobile phase.

The same macromer as in Example 1 was used. The gellable composition included macromer (7% w/w), AMPS (3%), Omnipaque 350 (50%), iron(III) citrate (2000 ppm) and hydrogen peroxide (500 ppm) in water. The viscosity of this solution was 43 cP. A 5 Fr, 100 cm catheter was used to deliver the gellable composition into the flow model.

The initiator was provided via the mobile phase in a solution including ascorbic acid (16.6 mM) in water. The flow rate was 80 ml/min.

Using a 3 ml syringe the macromer solution (3 ml) was injected through the catheter into the flow cell while the infusion solution was flowing. Upon injection an insoluble hydrogel mass was formed within the channels (3.00 g—75% recovery).

### Example 3

### Infusion of Initiator

The same macromer as in Example 1 was used. The gellable composition included macromer (7% w/w), AMPS (3%), Omnipaque 350 (50%), ascorbic acid (24.9 mM) and hydrogen peroxide (500 ppm) in water. The viscosity of this solution was 43 cP. A 5 Fr, 100 cm catheter was used to deliver the gellable composition into the flow model.

The initiator was provided via the mobile phase in a solution including iron (II) lactate (2000 ppm) in water. The flow rate was 80 ml/min.

Using a 3 ml syringe the macromer solution (3 ml) was injected through the catheter into the flow cell while the infusion solution was flowing. Upon injection an insoluble hydrogel mass was formed within the channels (3.10 g—79% recovery).

### Example 4

### Higher Flow Rate

The same conditions and compositions as in Example 3 were used; except that the flow rate of the infusion solution was 130 ml/min. 3.21 g hydrogel was formed—82% recovery.

### Example 5

### Aneurysm Model; Coaxial Catheter

This example uses a smaller 3.5×2.25 inch flow cell that contained a 0.37 inch circular aneurysm off the central flow channel.

The gellable composition included macromer (12% w/w), AMPS (6%), acetate buffer (100 mM) and hydrogen peroxide (500 ppm) in water. The viscosity was 47 cP. The initiator solution contained iron (II) lactate (2000 ppm) and ascorbic acid (8.3 mM) in water.

Using two Touy-Borst connectors, a 5 Fr guide catheter was placed approximately 1 inch from the aneurysm. A 3.0/2.3 Fr, 150 cm, Turbo Tracker 18 microcatheter was inserted into the aneurysm through the guide catheter. With water flowing through the flow cell at 100 ml/min, a 10 ml syringe was used to slowly infuse the iron lactate solution into the flow cell through the guide catheter. After 2.0 ml had been injected, the pre-polymer solution was simultaneously injected into the aneurysm using a 1 ml syringe connected to the microcatheter. After 0.5 ml of the prepolymer was injected a polymeric hydrogel had filled the aneurysm.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

1-17. (canceled)

18. An embolotherapy composition comprising a gellable composition and a solid article initiator.

19. The embolotherapy composition of claim 18, wherein the solid article initiator is one or more microspheres that are loaded with initiator.

20. The embolotherapy composition of claim 18, wherein the solid article initiator is a disk or coil.

21. The embolotherapy composition of claim 18, wherein the solid article initiator is a solid article having a gellation initiator coated on its surface, embedded therein, or impregnated therein.

22. The embolotherapy composition of claim 18, wherein the solid article initiator is a polymer sheet.

23. The embolotherapy composition of claim 18, wherein the solid article initiator is the tip of a catheter that delivers the gellable composition.

24. The embolotherapy composition of claim 18, wherein the gellable composition is macromers having crosslinkable groups that are crosslinkable via free radical polymerization.

25. The embolotherapy composition of claim 24, wherein the crosslinkable groups are olefinically unsaturated groups.

26. A method of embolotherapy comprising the steps:
placing a solid article initiator at the intended site of embolotherapy; and
delivering a gellable composition to the intended site of embolotherapy,
wherein the solid article initiator converts the gellable composition into a hydrogel.

27. The embolotherapy method of claim 26, wherein the solid article initiator is one or more microspheres that are loaded with initiator.

28. The embolotherapy method of claim 26, wherein the solid article initiator is a disk or coil.

29. The embolotherapy method of claim 26, wherein the solid article initiator is a solid article having a gellation initiator coated on its surface, embedded therein, or impregnated therein.

30. The embolotherapy method of claim 26, wherein the solid article initiator is a polymer sheet.

31. The embolotherapy method of claim 26, wherein the solid article initiator is placed at the site of intended embolotherapy prior to delivery of the gellable composition.

32. The embolotherapy method of claim 26, wherein the solid article initiator is placed at the site of intended embolotherapy at the same time as delivery of the gellable composition.

33. The embolotherapy method of claim 26, wherein the gellable composition is macromers having crosslinkable groups that are crosslinkable via free radical polymerization.

34. A method of embolotherapy using a composition that gels in response to contact with an initiator, comprising the steps:
providing a plurality of microspheres loaded with the initiator;
placing the plurality of microspheres at the intended site of embolotherapy;
delivering the composition to the intended site of embolotherapy so that it contacts the initiator and gels.

35. The method of claim 34, wherein the composition is a solution of macromers having pendant crosslinkable groups that are crosslinkable via free radical polymerization.
